(19) United States
(12) Patent Application Publication
(10) Pub. No.: US 2026/0165880 A1
(43) Pub. Date: Jun. 18, 2026

(54) OPHTHALMIC SURGICAL SYSTEM FOR TREATING AN EYE

(71) Applicant: CARL ZEISS MEDITEC AG, Jena (DE)

(72) Inventors: Max Wagenblast, Oberkochen (DE); Christoph Kübler, Oberkochen (DE)

(21) Appl. No.: 19/417,333

(22) Filed: Dec. 12, 2025

(30) Foreign Application Priority Data
Dec. 12, 2024 (DE) 10 2024 137 509.4

(51) Int. Cl. A61F 9/007

(52) U.S. Cl. CPC A61F 9/00736

(57) ABSTRACT

An ophthalmic surgical system for treating an eye has an aspiration-side fluid pump and a handpiece, coupled to the aspiration-side fluid pump, of which at least a portion is introducible into the eye with a needle tip. The ophthalmic surgical system can detect an occlusion at the needle tip and take at least one of the following measure to reduce the volume flow of aspiration fluid that occurs after an occlusion break: a volume flow limiting element is moved from a normal into a measure position and kept there, a volume flow limiting element is moved repeatedly back and forth between the normal and the measure position, and/or the aspiration fluid is guided through at least one flow restriction means which brings about a pressure drop in the volume flow downstream of the at least one flow restriction means in the case of an occlusion break, and so cavitation occurs.

### CROSS REFERENCE TO RELATED APPLICATIONS

This application claims priority to German patent application DE 10 2024 137 509.4, filed Dec. 12, 2024, the entire content of which is incorporated herein by reference.

### TECHNICAL FIELD

The disclosure relates to an ophthalmic surgical system for treating an eye. The disclosure also relates to a method for operating such an ophthalmic surgical system for treating a lens of an eye.

### BACKGROUND

An ophthalmic surgical system serves for treating an eye of a patient, for example within the scope of a cataract operation, in which the eye of the patient has a lens opacification, which is referred to as a cataract. Phacoemulsification is particularly widespread in this context; here, a thin needle, in particular a hollow needle, is introduced into a capsular bag of the eye to be treated and is excited to perform mechanical vibrations within the capsular bag. The needle, which is introduced into the eye in the region of a needle tip of the needle, is part of an ophthalmic surgical handpiece. The lens of the eye is comminuted with the aid of the vibrating needle tip, whereby lens particles are released in the eye. The released lens particles are aspirated via an aspiration line of the ophthalmic surgical system. At the same time, the eye is supplied with irrigation fluid via an irrigation line of the ophthalmic surgical system. As soon as the lens has been completely removed from the eye, a new artificial lens can be inserted into the emptied capsular bag. For example, the treated patient might achieve improved vision compared to their vision prior to the operation. As an alternative or in addition, the ophthalmic surgical system may be configured for use within the scope of a vitrectomy, in which a vitreous humor in the eye is comminuted with the ophthalmic surgical handpiece.

A situation in which a lens particle and/or a tissue blocks the aspiration line of the ophthalmic surgical system may arise during a use of said ophthalmic surgical system. Such a situation may be referred to as an occlusion of the needle tip of the handpiece of the ophthalmic surgical system. An increase in the intraocular pressure in the eye of the patient is observed immediately after the occurrence of the occlusion since the eye is still supplied with the irrigation fluid via the irrigation line. However, aspiration-side suctioning from the eye is prevented by the lens particle that caused the occlusion.

The lens particle blocking the aspiration line can be comminuted with the needle, which for example carries out vibrations in the ultrasonic wave range, whereupon the blockage of the aspiration line can be ended. This event may be referred to as occlusion break. After the occlusion break, an aspiration fluid with the lens particles can be abruptly aspirated from the eye via the aspiration line, inter alia assisted by a negative pressure applied to the aspiration side. This leads to an abrupt reduction in pressure in the eye, i.e., a fast and sudden reduction in the intraocular pressure. However, an eye collapse, for example, may occur if the intraocular pressure is reduced too much. The sudden reduction in the intraocular pressure following an occlusion break should thus at least be mitigated.

DE 10 2012 018 983 A1 describes an ophthalmic surgical apparatus for phacoemulsification, comprising an aspiration line, a suction vacuum pump and a flow limiter, which sets a flow through the aspiration line to an absolute value in the range of 5 milliliters per minute to 100 milliliters per minute. As seen in an aspiration direction, the flow limiter is arranged upstream of the suction vacuum pump.

WO 2022/034439 A1 describes a system and a method for attenuating a volume flow of aspiration fluid following an occlusion. A valve is fluid-connected to a suction line and a container for the aspiration fluid and provides one or more channels between the suction line and the container. A computer controls the valve on the basis of a sensed pressure in order to mitigate a change in pressure or volume.

EP 2 164 435 B1 describes a system for reversing a collapse of an ocular chamber following an occlusion for surgical equipment, which generates a response comprising temporarily blocking the distal end of the aspiration path in order to terminate the ocular chamber collapse and simultaneously temporarily venting the aspiration line to relieve the vacuum.

U.S. Pat. No. 7,083,591 B2 describes a pressure flow regulator for an ophthalmic surgical instrument, which has an infusion line and an aspiration line. The pressure flow regulator comprises a flow limiting apparatus fluid-connected to the aspiration line in order to control the pressure drop on account of aspirated fluid.

US 2023/0181359 A1 describes a system for controlling the aspiration of a phacoemulsification system having an apparatus which is coupled to an aspiration line of the phacoemulsification system in order to regulate the flow in the aspiration line and a pressure wave damper which is fluid-connected to the aspiration line and contains an elastic element. The elastic element is configured to experience a change in shape in response to a pressure pulse generated by the apparatus so as to suppress an amplitude of the pressure wave before the pressure pulse reaches the eye.

### SUMMARY

It is an object of the disclosure to provide a solution with which an intraocular pressure drop in an eye of a patient after an occlusion break can be reduced.

The object is achieved by an ophthalmic surgical system and a method for operating the ophthalmic surgical system as described herein.

A first aspect of the disclosure relates to an ophthalmic surgical system for treating an eye of a patient. The ophthalmic surgical system includes at least one console including at least one portion of at least one aspiration-side fluid pump for conveying an aspiration fluid. Moreover, an exemplary embodiment of the ophthalmic surgical system may include at least one portion of at least one irrigation-side fluid pump for conveying a treatment fluid. Alternatively, the treatment fluid may be referred to as irrigation fluid. In a typical example, the ophthalmic surgical system includes two irrigation-side fluid pumps and two aspiration-side fluid pumps.

Moreover, the ophthalmic surgical system includes an ophthalmic surgical handpiece for treating the eye. At least a portion of the ophthalmic surgical handpiece is introducible into the eye with a needle tip of the handpiece. The ophthalmic surgical handpiece is coupled at least indirectly to the at least one aspiration-side fluid pump. It may also be coupled at least indirectly to the at least one irrigation-side fluid pump. A needle-tip-including needle of the handpiece is configured as a hollow needle such that the aspiration fluid can be aspirated from the eye through the needle. The treatment fluid, by contrast, is for example guided into the eye via a sleeve that surrounds the hollow needle. The sleeve has a passage opening with a diameter which is larger than an outer diameter of the hollow needle and from which the hollow needle projects. In this case, the treatment fluid is guided into the eye via a fluid channel which is formed between an outer side of the hollow needle and an inner side of the sleeve, and which ends at the passage opening. Alternatively, the sleeve includes a separate opening for the treatment fluid in addition to the passage opening. The sleeve can be introduced into the eye together with the hollow needle in the region of the needle tip. The ophthalmic surgical handpiece includes two fluid lines that are separated from each other, one for the treatment fluid and one for the aspiration fluid, and so two mutually separate hydraulic connections may be formed to the eye of the patient.

For example, the console may include a cassette accommodation region for accommodating a cassette. In this case, the at least one irrigation-side fluid pump and/or the at least one aspiration-side fluid pump may be at least partially included by the cassette. For example, the cassette may be connected directly to the ophthalmic surgical handpiece via a supply line. As an alternative exemplary embodiment, the console may be connected directly to the ophthalmic surgical handpiece via the supply line. For example, the supply line includes at least one portion of an aspiration line for the aspiration fluid. Moreover, the supply line may include at least one portion of an irrigation line for the treatment fluid. In this example, the handpiece has two lines that are separate from each other. As an alternative, the handpiece may include a double line for the treatment fluid and the aspiration fluid.

In other words, the ophthalmic surgical system includes an aspiration side. It may also include an irrigation side. On the irrigation side, the at least one irrigation-side fluid pump can be used to pump the treatment fluid from a treatment fluid storage vessel, which may be arranged outside the console of the ophthalmic surgical system, to the handpiece and, from the latter, into the eye of the patient. Furthermore, on the aspiration side, the aspiration fluid can be pumped out of the eye to an aspiration fluid collection vessel via the handpiece with the at least one aspiration-side fluid pump. Alternatively, the aspiration fluid collection vessel may be referred to as drainage container.

In particular, the ophthalmic surgical system is configured to detect an occlusion at the needle tip by applying an occlusion detection algorithm. An occlusion is an event or a situation in which at least one access to the aspiration line of the ophthalmic surgical system is obstructed at the needle tip of the handpiece. For example, a lens particle in the eye may at least temporarily close and thus block an entrance of the aspiration line of the handpiece. The occlusion detection algorithm includes at least one rule and/or instruction which, when carried out, detects the occlusion, for example on the basis of measurement data provided by the ophthalmic surgical system. In this context, an example allows for the use of known methods for detecting an occlusion at the needle tip of a handpiece.

The ophthalmic surgical system is configured to take at least one measure after the detection of the occlusion in order to reduce a volume flow of aspiration fluid from the eye to the at least one aspiration-side fluid pump that occurs after the detected occlusion breaks, in comparison with the volume flow of aspiration fluid without a measure being taken. This achieves a smaller intraocular pressure drop in the eye of the patient following the occlusion break since, in comparison with the case of not taking any measure, less aspiration fluid is aspirated from the eye per unit time and hence a smaller volume flow of aspiration fluid is aspirated from the eye with the at least one aspiration-side fluid pump. The pressure drop in the intraocular pressure caused on account of the occlusion break is thus mitigated by taking the at least one measure.

The ophthalmic surgical system is configured to take at least one of following measures as the at least one measure: The following distinguishes between three measures, which may be referred to as measure a, measure b and measure c. Should measure a be taken, the ophthalmic surgical system is configured to move at least one volume flow limiting element from a normal position into a measure position and keep the latter in the measure position. In the normal position, a portion of the ophthalmic surgical system through which the aspiration fluid can flow has a first cross section between the handpiece and the at least one aspiration-side fluid pump. Hereinafter, the portion of the ophthalmic surgical system through which the aspiration fluid can flow always means the portion of the ophthalmic surgical system, though which the aspiration fluid can flow, between the handpiece and the at least one aspiration-side fluid pump. The portion of the ophthalmic surgical system through which the aspiration fluid can flow may be understood as an aspiration line of the ophthalmic surgical system through which the aspiration fluid can flow and in particular does flow. The portion of the ophthalmic surgical system through which the aspiration fluid can flow may in one exemplary embodiment include an element which is integrated in the aspiration line and through which the aspiration fluid can flow and in particular does flow.

In the measure position, the portion of the ophthalmic surgical system through which the aspiration fluid can flow, in particular the aspiration line, has at least locally a second cross section that is reduced in comparison with the first cross section. Thus, the cross section of the portion of the ophthalmic surgical system through which the aspiration fluid can flow, i.e., the cross section of the aspiration line in a typical example, is reduced if measure a is taken. In a typical example, the first cross section is a cross section that is unaffected by the volume flow limiting element. For example, the first cross section may correspond to the cross section of the aspiration line without external influence. In an exemplary embodiment, the aspiration line may be configured at least partially as a tube which, in the normal position of the at least one volume flow limiting element, has the first cross section and, in the measure position, has the second cross section at least locally.

In an exemplary embodiment of the disclosure, the at least one volume flow limiting element may be configured as a valve capable of at least partially blocking the portion of the ophthalmic surgical system through which the aspiration fluid can flow between the handpiece and the at least one aspiration-side fluid pump, in particular the aspiration line. For example, the valve may be an inlet valve of the at least aspiration-side fluid pump or an additional valve of the ophthalmic surgical system. As an alternative or in addition, the at least one volume flow limiting element may be configured as an element which presses against the aspiration line from the outside and which, for example in a locally limited manner, presses the aspiration line together and thereby locally reduces the cross section thereof. As an alternative or in addition, the at least one volume flow limiting element can be configured as an element which can be moved into the aspiration line, for example as a stop, flap or any other component.

The second cross section is predetermined on the basis of the handpiece and/or a pressure in the at least one aspiration-side fluid pump. For example, depending on the diameter of the needle tip of the handpiece, a second cross section adapted to this diameter may be chosen. For example, in the case of a needle tip with a small diameter, the second cross section may be chosen to be larger than in the case of a needle tip with a larger diameter. Thus, the cross section is reduced to a larger extent during the change into the measure position should the needle tip lead to a small pressure loss, in comparison with a needle tip with a larger pressure loss. Thus, a different second cross section for the measure position may be predetermined depending on the choice of handpiece. As an alternative or in addition, the at least one aspiration-side fluid pump may have a different configuration, and therefore a negative pressure that can be set on the aspiration side may have different magnitudes during the occlusion. For example, should a high negative pressure and hence a low pressure be set in the at least one aspiration-side fluid pump during the occlusion, a small second cross section is chosen in the measure position, whereas a larger second cross section is predetermined during the occlusion in the event of a comparatively lower negative pressure and thus higher pressure in the at least one aspiration-side fluid pump. The pressure that can be set in the at least one aspiration-side fluid pump may therefore influence the second cross section.

As an alternative or in addition, the ophthalmic surgical system may be configured to take measure b and then move at least one volume flow limiting element repeatedly back and forth between the normal position and the measure position. The volume flow limiting element used to this end may have an analogous configuration to the above-described volume flow limiting element. Should the ophthalmic surgical system include exactly one volume flow limiting element, it is possible to take either measure a or measure b at a predetermined time. The normal position and/or measure position described in connection with measure a may correspond to the normal position and measure position, respectively, provided for measure b. The measure position according to measure a may deviate from the measure position according to measure b. Thus, in an exemplary embodiment, a different second cross section may be predetermined for measure b than for measure a. In an exemplary embodiment, the second cross section is 0 in measure b, i.e., the aspiration line is for example completely closed-off at least locally, and hence the volume flow of aspiration fluid in the direction of the at least one aspiration-side fluid pump is completely suppressed in the measure position.

The repeated back-and-forth movement between the normal position and the measure position is implemented at a frequency which describes the time intervals at which there is a change between the normal position and the measure position. Measure b thus provides for the cross section of the portion of the ophthalmic surgical system through which the aspiration fluid can flow between the handpiece and the at least one aspiration-side fluid pump, in particular the aspiration line, to be repeatedly reduced at least locally at predetermined time intervals and to subsequently be increased again. Overall, this leads to a reduction in the volume flow after the occlusion break since the cross section of the aspiration line is repeatedly made smaller or even reduced to 0. Thus, what can be achieved as a result of measure b is that the intraocular pressure drop in the eye following the occlusion break is mitigated.

As an alternative to that or in addition, the ophthalmic surgical system may be configured to guide the aspiration fluid through at least one flow restriction means as measure c. The flow restriction means is arranged between the needle tip of the handpiece and the at least one aspiration-side fluid pump. For example, the flow restriction means is situated in or along the aspiration line. The flow restriction means is configured to bring about a pressure drop in the volume flow of aspiration fluid downstream of the at least flow restriction means, as seen in the flow direction of the aspiration fluid, in the case of an occlusion break, with cavitation occurring at the brought-about pressure drop. In this case, cavitation means the formation of bubbles in the aspiration fluid should a local pressure of the aspiration fluid fall below the vapor pressure of the aspiration fluid. As a result, a vapor bubble forms in the aspiration fluid and generally lasts only for a short time before it collapses again into the aspiration fluid. The cavitation reduces the volume flow, i.e., it achieves mitigation of the intraocular pressure drop in the eye after the occlusion break since less aspiration fluid can be aspirated than in the case of an ophthalmic surgical system without a flow restriction means. To ensure that the cavitation is possible as described, the aspiration fluid should be understood to be a liquid in which individual lens particles are situated. At least in measure c, the aspiration fluid may alternatively be referred to as an aspiration liquid.

Thus, three different measures have been provided, which may be taken individually or combined with one another and in each case contribute to reducing the volume flow of aspiration fluid away from the eye after the occlusion break, whereby it is for example possible to ensure that the intraocular pressure in the eye does not drop below a predetermined minimum intraocular pressure. For example, the at least one measure may be started immediately as soon as the occlusion has been detected, and this measure may be continued at least until the occlusion break is detected. Thus, the at least one measure is already started at a time at which the occlusion break has not yet occurred. As soon as the occlusion break occurs and still before the latter is detected with the ophthalmic surgical system, the at least one measure is thus already performed and hence has already been taken at the time of the occlusion break.

An exemplary embodiment provides that, when measure a is taken, the second cross section is predetermined in such a way that, in the measure position, the portion of the ophthalmic surgical system through which the aspiration fluid can flow between the handpiece and the at least one aspiration-side fluid pump, in particular the aspiration line, is closed at least locally by 20 percent to 100 percent in comparison with the normal position. A degree of closure of the portion through which fluid can flow, in particular of the aspiration line, attained in the measure position, may thus be 20 percent to 100 percent of the cross section without any measure being taken and hence of the first cross section in the normal position. In particular, 70 percent to 80 percent of the portion though which fluid can flow, in particular the aspiration line, may be closed at least locally. In other words, the second cross section is between 80 percent and 0 percent, in particular between 30 percent and 10 percent, of the first cross section. From this it is clear that measure a allows for the aspiration flow to be reduced only slightly or even for it to be suppressed entirely. The specified degrees of closure are suitable for influencing the volume flow depending on the handpiece and/or the pressure in the at least one aspiration-side fluid pump to such an extent that an unwanted pressure drop in the intraocular pressure can always be prevented after the occlusion break.

Should the degree of closure be 100 percent and hence the volume flow be completely suppressed after the occlusion break, an assumption made in one example is that a negative pressure has been set within the aspiration line between the at least one volume flow limiting element and the needle tip of the handpiece, for example by way of appropriately controlling the at least one aspiration-side fluid pump before the volume flow limiting element is moved into the measure position, so that a suction pressure acts, the latter for example at least temporarily driving the aspiration of the aspiration fluid from the eye following the comminution of the lens particle causing the occlusion. In this example, the at least one volume flow limiting element can be moved back into the normal position after the occlusion break is detected so that the aspiration fluid can be moved as far as the aspiration fluid collection vessel.

An exemplary embodiment for the case where measure b is taken provides for the ophthalmic surgical system to be configured to repeatedly move the at least one volume flow limiting element between the normal position and the measure position in such a way that it is alternately arranged in the normal position for a predetermined first time period and, thereafter, in the measure position for a predetermined second time period. The first time period and the second time period may be the same or differ from each other. Thus, the length of time for which the measure position and the normal position are provided in each case may be fixedly provided, and this gives rise to the length of time that a position is adopted and ultimately the number of changes between the normal position and the measure position being predetermined. Thus, a cyclically repeating opening and closing cycle of the volume flow limiting element may be described on the basis of the first time period and the second time period. In an exemplary embodiment, the first time period is chosen to be shorter than the second time period such that the volume flow limiting element is in the measure position for the same amount of time in each case, whereby it is possible to achieve a significant reduction in the volume flow of aspiration fluid in comparison with a longer first time period.

According to an additional exemplary embodiment, the predetermined first time period and the predetermined second time period are predetermined on the basis of the handpiece and/or a pressure in the at least one aspiration-side fluid pump. Hence, the same dependencies as for measure a may also be provided for measure b. Not only the second cross section but also the first and the second time period may therefore be selected on the basis of the chosen handpiece and/or the pressure in the aspiration-side fluid pump. This allows a dynamic adaptation to the ophthalmic surgical system used.

Moreover, in the case of measure c being taken, an exemplary embodiment provides for the flow restriction means to be arranged in such a way that it is arranged in the handpiece and/or in the aspiration line. As an alternative or in addition, the flow restriction means is arranged in the console. If arranged in the console, it is arranged upstream of the least a portion of the at least one aspiration-side fluid pump, as seen in the flow direction of the aspiration fluid. As an alternative or in addition, the flow restriction means may be arranged in the cassette of the ophthalmic surgical system, with the cassette including at least one portion of the at least one aspiration-side fluid pump. In this case, the flow restriction means is arranged upstream of the at least a portion of the at least one aspiration-side fluid pump, as seen in the flow direction of the aspiration fluid. The flow restriction means may be positioned at any desired location between the needle tip and the aspiration-side fluid pump. The flow restriction means can be integrated into the ophthalmic surgical system in many different ways.

For example, the flow restriction means may include an add-on component which may be installed in the ophthalmic surgical system on a permanent or temporary basis. For example, the aspiration line may be separated into two line sections which are spatially separated from each other, and the add-on component having the flow restriction means may be arranged between the two line sections, with the add-on component interconnecting the two line sections.

Another exemplary embodiment in which measure c was taken provides for the ophthalmic surgical system to include at least one diverting element. The ophthalmic surgical system is configured to guide the aspiration fluid through a first partial flow path of the ophthalmic surgical system, in which the flow restriction means is arranged, by virtue of controlling the at least one diverting element when measure c is taken. Moreover, the ophthalmic surgical system is configured to guide the aspiration fluid in a second partial flow path of the ophthalmic surgical system which differs from the first partial flow path and in which no flow restriction means is arranged, by virtue of controlling the at least one diverting element when measure c is not taken. Thus, the aspiration line is divided or split among the two partial flow paths, with a change between the two partial flow paths being able to be brought about with the at least one diverting element. The two partial flow paths may be understood to be two sections of the aspiration line that are spatially separated from each other. For example, the diverting element is configured as a directional control valve or any other component which can guide a volume flow in different directions.

In surgical operation without an occlusion event of the ophthalmic surgical system, during which measure c is not taken, the aspiration fluid is not guided through the flow restriction means. For example, this may prevent a lens particle from becoming stuck in the region of the flow restriction means and blocking the aspiration line while the lens particles are aspirated from the eye. In this example, aspiration fluid only ever flows though the flow restriction means if, after the occlusion has been detected, measure c has been taken, and the occlusion break has occurred, i.e., when the described reduction in the volume flow is desired. The described implementation of measure c thus leads to the surgical operation not being influenced by the flow restriction means, whereby it is particularly stable despite the flow restriction means being provided.

The surgical operation without an occlusion event means operation or use of the ophthalmic surgical system at a point in time before the occlusion and after the occlusion, after the occlusion break has taken place and the intraocular pressure drop triggered by the occlusion break has been compensated for, such that the intraocular pressure in the eye has been set to a predetermined target intraocular pressure or substantially to the target intraocular pressure. In this case, substantially is intended to encompass deviation from a predetermined target intraocular pressure of up to 0.5 percent, 1 percent, 2 percent, 3 percent, 5 percent, or 10 percent.

For example, the target intraocular pressure is specified in a manner specific to the patient, by a treating person, such as a surgeon or any other member of medical staff, before the start of or during the treatment of the eye. As an alternative or in addition, the target intraocular pressure may be determined and provided, in particular may be predetermined automatically, with the ophthalmic surgical system. The target intraocular pressure may alternatively be referred to as a set intraocular pressure (IOP) value. The predetermined target intraocular pressure is the intraocular pressure that should typically be kept constant during the treatment of the eye and hence during the use of the ophthalmological system. Since variations in the intraocular pressure occur during the occlusion and after the occlusion break, the intraocular pressure deviates at least temporarily from the target intraocular pressure. However, during the surgical operation without an occlusion event, the intraocular pressure typically corresponds to the target intraocular pressure.

Moreover, the assumption may be made that the ophthalmological system is typically operated during the occlusion in such a way that controlling the at least one irrigation-side fluid pump sets the target intraocular pressure in the eye. For example, should the intraocular pressure initially rise after the onset of the occlusion since treatment fluid is still supplied to the eye without aspiration fluid being able to be aspirated, appropriate control of the at least one irrigation-side fluid pump causes the intraocular pressure to be reduced again until it has reached the target intraocular pressure, and the latter is subsequently kept constant. As an alternative, the at least one irrigation-side fluid pump may be controlled in such a way that an intraocular pressure corresponding to the target intraocular pressure plus a predetermined target intraocular pressure increase is set, and so a higher intraocular pressure than the target intraocular pressure is set temporarily. Independently of which intraocular pressure is set after the occlusion is detected, it is possible to take at least one of measures a, b, and/or c in order to reduce the intraocular pressure drop following the occlusion break.

A further exemplary embodiment in which measure c is taken provides for the flow restriction means to include a movable stop. The flow restriction means with the movable stop is configured to move the movable stop into a predetermined position, in which the cavitation occurs in the case of the pressure drop brought about by the occlusion break when said occlusion break occurs, when measure c is taken. For example, the movable stop may be completely retracted or pulled back whenever measure c is not taken such that the cross section of the aspiration line in the region of the flow restriction means, for example, corresponds to the first cross section in the normal position. The flow restriction means may be configured to keep the movable stop in an initial position or normal position, which deviates from the predetermined position and in which the stop is completely retracted or pulled back, whenever measure c is not taken.

By moving the stop into the predetermined position, the cross section of the aspiration line, a cross section of the line for the aspiration fluid in the handpiece and/or a cross section of the line for the aspiration fluid in the console or cassette can be locally reduced, and so the flow restriction means is configured such that the pressure drop in the volume flow takes place in such a way in the case of the occlusion break that the cavitation occurs and hence the desired reduction in the volume flow is achieved. The movable stop is advantageous in that the aspiration fluid flows through the flow restriction means only when the latter is required since the occlusion was detected and the occlusion break is expected soon, and not during surgical operation without an occlusion event. Although this leads to a complex configuration of the flow restriction means, it is possible to rule out influencing the volume flow of aspiration fluid during the surgical operation without an occlusion event.

According to a further exemplary embodiment, the ophthalmic surgical system is configured, after the detection of the occlusion, to control the at least one aspiration-side fluid pump in such a way that a negative pressure, in particular a maximum possible negative pressure, sets in in the at least one aspiration-side fluid pump. After the occlusion is detected, the at least one aspiration-side fluid pump thus is controlled in such a way that a suction force that is as high as possible occurs at the needle tip, for example in order to attempt to aspirate the lens particle causing the occlusion and/or aspirate the latter after it was comminuted. If the maximum possible negative pressure is set, the greatest possible suction force is obtained at the needle tip, for example. However, the negative pressure in the at least one aspiration-side fluid pump leads to a comparatively large volume flow of aspiration fluid initially being aspirated after the occlusion break and hence being removed from the eye, as a result of which the undesirably large intraocular pressure drop in the eye is caused in turn. Nevertheless, control with negative pressure on the part of the aspiration-side fluid pump is necessary in order to terminate the occlusion as quickly as possible and to aspirate the lens particle causing the occlusion out of the eye.

Moreover, the ophthalmic surgical system is configured to control the at least one aspiration-side fluid pump and/or the at least one irrigation-side fluid pump of the ophthalmic surgical system in such a way after the detection of the occlusion break that a target intraocular pressure in the eye, as predetermined for a surgical operation without an occlusion event, sets in. In particular, at least taken measures a and/or b are terminated no later than the target intraocular pressure being reached. At least taken measures a and/or b being terminated means that at least measures a and/or b are terminated and that measure c may additionally also be terminated. In the case of measure c, the latter is for example terminated by virtue of the movable stop being retracted or pulled back and/or by virtue of the diverting element being controlled in such a way that the aspiration fluid is guided through the second partial flow path. Should the flow restriction means be configured in such a way that it is positioned permanently between the needle tip and the at least one aspiration-side fluid pump and aspiration fluid is guided therethrough, the assumption can be made that there is no cavitation in the case of a volume flow of aspiration fluid that can arise during the surgical operation, and so measure c may be considered to be terminated. When the flow restriction means is positioned between the needle tip and the at least one aspiration-side fluid pump, said flow restriction means may be a portion of the needle in an example.

For example, if measure a is implemented in such a way that the second cross section is 0, i.e., a degree of closure of 100 percent is predetermined, then an example may provide for measure a to already be terminated directly after the occlusion break is detected, i.e., before the target intraocular pressure is reached. Here, the assumption is made that the occlusion break is always detected after said occlusion break occurs. Further, in the case of a degree of closure of 100 percent after the detection of the occlusion break, the at least one volume flow limiting element can be at least partially reopened, i.e., a further measure position between the normal position and the measure position in which the second cross section is equal to 0 can be adopted. This further measure position may be referred to as an intermediate position between the normal position and the measure position. In the further measure position or intermediate position, aspiration fluid may be aspirated with the at least one aspiration-side fluid pump. In other words, the respective measure may be terminated any time after the occlusion break is detected, with the measure only being terminated after the target intraocular pressure has been reached in a typical example. From this it is clear that the respective measure a, b, and/or c does not remain activated permanently but is only provided temporarily such that the volume flow of aspiration fluid is not intended to be influenced during the surgical operation without an occlusion event.

Furthermore, an exemplary embodiment provides for the ophthalmic surgical system to be configured to apply the occlusion detection algorithm to movement information in order to detect the occlusion. The movement information describes a current movement of a separation element between a pump chamber and a drive chamber of the at least one aspiration-side fluid pump. As an alternative to that or in addition, the movement information may describe a current movement of a separation element between a pump chamber and a drive chamber of the at least one irrigation-side fluid pump of the ophthalmic surgical system in one example. In a typical example, the movement information describes the current movement of the separation element in the at least one aspiration-side fluid pump. For example, the movement information is determined with a position sensor associated with the fluid pump in question. In that case, the occlusion is detected at least on the basis of the movement information being smaller than a predetermined movement limit value. The movement limit value may be predetermined in such a way that the separation element no longer moves or substantially no longer moves. In this case, substantially is intended to encompass deflections of the separation element of up to 1 percent, 2 percent, 3 percent, 5 percent, or 10 percent, in relation to a maximum possible deflection. The lack of a separation element movement may thus indicate that the occlusion has occurred since, for example, no aspiration-side movement of the separation element can be observed during said occlusion.

As an alternative or in addition, the ophthalmic surgical system is configured to apply an occlusion break detection algorithm to the movement information described above in order to detect the occlusion break and detect the occlusion break at least on the basis of the movement information being larger than a predetermined movement limit value. In this case, the at least one aspiration-side fluid pump may be considered. Thus, whenever a movement in the at least one fluid pump is detected, the inference is made that the occlusion has broken, and the ophthalmic surgical system is capable of pumping aspiration fluid out of the eye again. The occlusion break detection algorithm may correspond at least in part to the occlusion detection algorithm or may differ from the latter. For example, the occlusion break detection algorithm includes at least one rule and/or instruction which, when carried out, detects the break and hence the end of the occlusion detected previously.

The two movement limit values mentioned here may be the same or differ from each other. For example, the predetermined movement limit value for detecting the occlusion may be chosen to be smaller than the predetermined movement limit value for detecting the occlusion break, or vice versa. Only the at least one aspiration-side fluid pump is considered in a typical example.

Thus, the occlusion and the occlusion break can be detected reliably and quickly in this way of the basis of the separation element movement, and the information gathered in the process can be processed reliably and quickly as well. As soon as the occlusion break has been detected, the measure taken is for example withdrawn or no longer accepted, and the ophthalmic surgical system is controlled in such a way that the intraocular pressure is regulated to the predetermined target intraocular pressure and set.

A further aspect of the disclosure relates to a method for operating an ophthalmic surgical system, having at least: a console including at least one portion of at least one aspiration-side fluid pump for conveying an aspiration fluid, and an ophthalmic surgical handpiece, at least a portion of said handpiece being introducible into an eye with a needle tip of a hollow needle of the handpiece and said handpiece being coupled at least indirectly to the aspiration-side fluid pump. In an example, the method is performed during treatment of an eye, for example during cataract surgery or vitreous humor surgery, i.e., a vitrectomy. The method should not be understood as a treatment method but as a method the performs calculations in order to determine and implement a control command for the ophthalmic surgical system, with the control command relating to at least one of three measures a, b and c.

In particular, the method includes detecting an occlusion at the needle tip by applying an occlusion detection algorithm. After the occlusion is detected, at least one measure is taken in order to reduce a volume flow of aspiration fluid from the eye to the at least one aspiration-side fluid pump that occurs after the detected occlusion breaks, in comparison with the volume flow of aspiration fluid without a measure being taken. At least one of the following measures is taken as the at least one measure:

Measure a includes moving at least one volume flow limiting element from a normal position into a measure position and keeping the latter in the measure position. A portion of the ophthalmic surgical system through which the aspiration fluid can flow between the handpiece and the at least one aspiration-side fluid pump has, in the normal position, a first cross section and, in the measure position, a second cross section that is at least locally reduced in comparison with the first cross section, the second cross section being predetermined on the basis of the handpiece and/or a pressure in the at least one aspiration-side fluid pump. The part of the ophthalmic surgical system through which the aspiration fluid can flow between the handpiece and the at least one aspiration-side fluid pump may for example be formed by an aspiration line between the handpiece and the at least one aspiration-side fluid pump.

Measure b includes repeatedly moving at least one volume flow limiting element back and forth between the normal position and a measure position, for example the measure position chosen within the scope of measure a.

Measure c includes guiding the aspiration fluid through at least one flow restriction means arranged between the needle tip of the handpiece and the at least one aspiration-side fluid pump, with the flow restriction means bringing about, in the case of the occlusion break, a pressure drop in the volume flow of aspiration fluid downstream of the at least one flow restriction means, as seen in the flow direction of the aspiration fluid, with a cavitation occurring in the case of the brought-about pressure drop.

For example, the method is performed with a control apparatus of the ophthalmic surgical system. The control apparatus includes a processor device. The latter may include at least one microprocessor, microcontroller, a field programmable gate array (FPGA) and/or a digital signal processor (DSP).

The exemplary embodiments described in connection with the ophthalmic surgical system according to the disclosure, in each case individually and in combination with one another, apply, mutatis mutandis and if applicable, to the method according to the disclosure. The disclosure includes combinations of the exemplary embodiments described.

### BRIEF DESCRIPTION OF THE DRAWINGS

The disclosure will now be described with reference to the drawings wherein:

FIG. 1 shows a schematic illustration of an ophthalmic surgical system;

FIG. 2 shows a schematic illustration of fluid flows in an ophthalmic surgical system prior to an occlusion;

FIG. 3 shows a schematic illustration of fluid flows within an ophthalmic surgical system during an occlusion;

FIG. 4 shows a schematic illustration of the flow speed on the irrigation side and aspiration side before and during an occlusion;

FIG. 5 shows a schematic illustration of a typical profile of an irrigation-side and aspiration-side fluid pump pressure or control air pressure before, during and after an occlusion;

FIG. 6 shows a schematic illustration of a profile of the intraocular pressure according to a standard procedure according to an exemplary embodiment of the disclosure;

FIG. 7 shows a schematic illustration of a fluid pump of an ophthalmic surgical system;

FIG. 8 shows an example of an implementation of a measure a,

FIG. 9 shows a temporal profile of changes between a normal position and a measure position according to a measure b;

FIG. 10 shows a relationship between a volume flow and an aspiration-side pressure in measure a or b;

FIG. 11 shows a schematic illustration of a flow restriction means for a measure c;

FIG. 12 shows an example of an arrangement of a flow restriction means in a handpiece;

FIG. 13 shows a schematic illustration of a diverting element for a measure c;

FIG. 14 shows a schematic illustration of a predetermined position of a diverting element according to FIG. 13; and

FIG. 15 shows a schematic illustration of a flow restriction means having a movable stop.

### DESCRIPTION OF EXEMPLARY EMBODIMENTS

In the figures, functionally identical elements are given the same reference numerals.

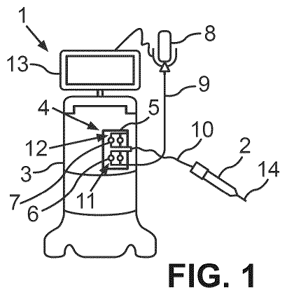

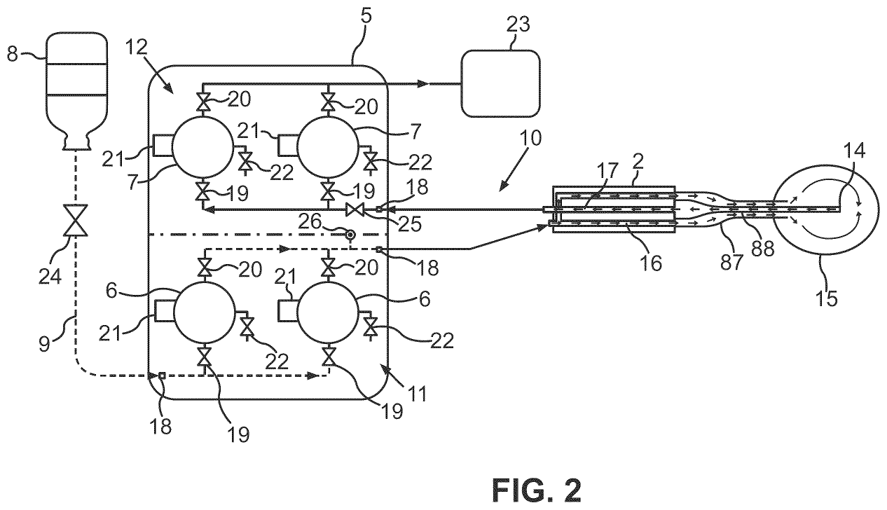

FIG. 1 shows an ophthalmic surgical system 1. The ophthalmic surgical system 1 includes an ophthalmic surgical handpiece 2 which is suitable, with a needle tip 14, for insertion into an eye 15 (see reference numeral 15 in FIG. 2) of a patient to be treated within the scope of an operation, for example a cataract operation. The ophthalmic surgical system 1 includes a console 3. The latter for example includes a cassette accommodation region 4 for a cassette 5 of the ophthalmic surgical system 1. The console 3 and/or the cassette 5 for example includes at least one portion of at least one irrigation-side fluid pump 6 for conveying a treatment fluid 16 (see reference numeral 16 in FIG. 2), the latter for example being able to be provided in a treatment fluid storage vessel 8 and be guided via a treatment fluid line 9 to the console 3 or to the cassette 5. The console 3 and/or the cassette 5 for example includes at least one portion of at least one aspiration-side fluid pump 7 for conveying an aspiration fluid 17 (see reference numeral 17 in FIG. 2).

There may be a supply line 10 present between the console 3 or the cassette 5 and the ophthalmic surgical handpiece 2, said supply line for example allowing the treatment fluid 16 to be guided to the handpiece 2 and/or allowing the aspiration fluid 17 to be guided from the handpiece 2 to the console 3 or cassette 5. The cassette 5 may therefore include, for example, an irrigation side 11 for pumping the treatment fluid 16 in the direction of the handpiece 2 and an aspiration side 12 for aspirating the aspiration fluid 17 from the eye 15.

The ophthalmic surgical system 1 may further include a display device and/or user interface 13, which for example can display information to a person treating the patient.

FIG. 2 shows fluid flows within the ophthalmic surgical system 1 before occlusion. It becomes clear that the handpiece 2 is inserted into the eye 15 of the patient with the needle tip 14 while the ophthalmic surgical system 1 is used for treating the eye 15. Moreover, details of the cassette 5 and of the fluid pumps 6, 7 encompassed thereby are sketched out in this case as an example. It becomes clear that numerous valves 19, 20, 22 may be arranged in the cassette 5 both on the irrigation side 11 and on the aspiration side 12. Respective inlet valves 19 for admitting into the associated fluid pump 6, 7 and outlet valves 20 for discharging the treatment fluid 16 and aspiration fluid 17, respectively, from the associated fluid pump 6, 7 are sketched out in this case. Furthermore, a compressed-air valve 22 for a compressed-air supply into the respective fluid pump 6, 7 may be present. A deflection 85 (see reference numeral 85 in FIG. 7) and hence a movement of a separation element 82 (see reference numeral 82 in FIG. 7) of the respective fluid pump 6, 7 may be detected with a position sensor 21. Furthermore, a pressure sensor 26 may be provided in the cassette 5. Provision can be made for an aspiration fluid collection vessel 23, into which the aspiration fluid 17 is guided.

A clamp 24 is also shown; it can be used to control the inflow of the treatment fluid 16 from the treatment fluid storage vessel 8 in the direction of the console 3. Furthermore, ports 18 are plotted, with which access may be provided to the treatment fluid storage vessel 8 or to the handpiece 2. Furthermore, a further valve 25 is also shown on the aspiration side 12.

It becomes clear that the treatment fluid 16 flows from the cassette 5 to the eye 15 through the handpiece 2. Moreover, the aspiration fluid 17 is aspirated from the eye 15 in the direction of the cassette 5. The tubes used to this end for the treatment fluid 16 and the aspiration fluid 17 together form the supply line 10. Within the handpiece 2, the treatment fluid 16 may be guided through a sleeve 87 which surrounds a hollow needle 88 through which the aspiration fluid 17 is suctioned.

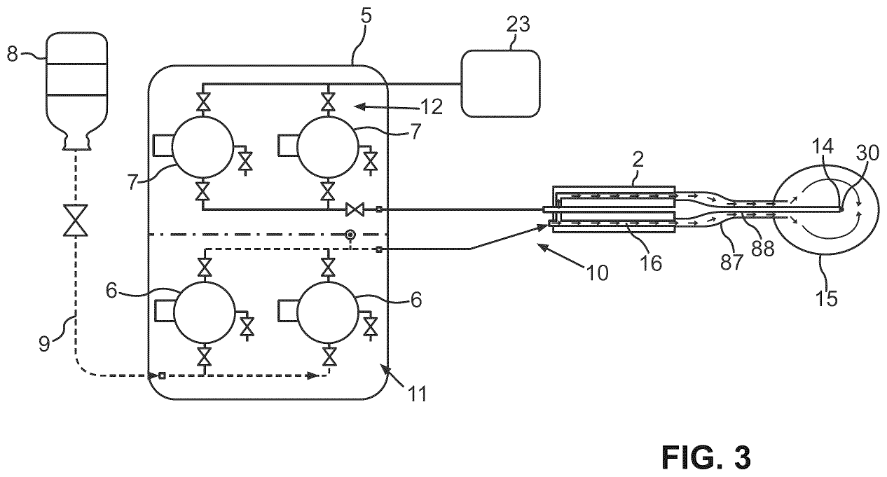

FIG. 3 shows the same construction as in FIG. 2, albeit for the case that a lens particle 30 blocks the needle tip 14 of the handpiece 2, with the result that aspiration fluid 17 is no longer able to be aspirated from the eye 15. However, it is still possible to supply treatment fluid 16. This situation is referred to as an occlusion.

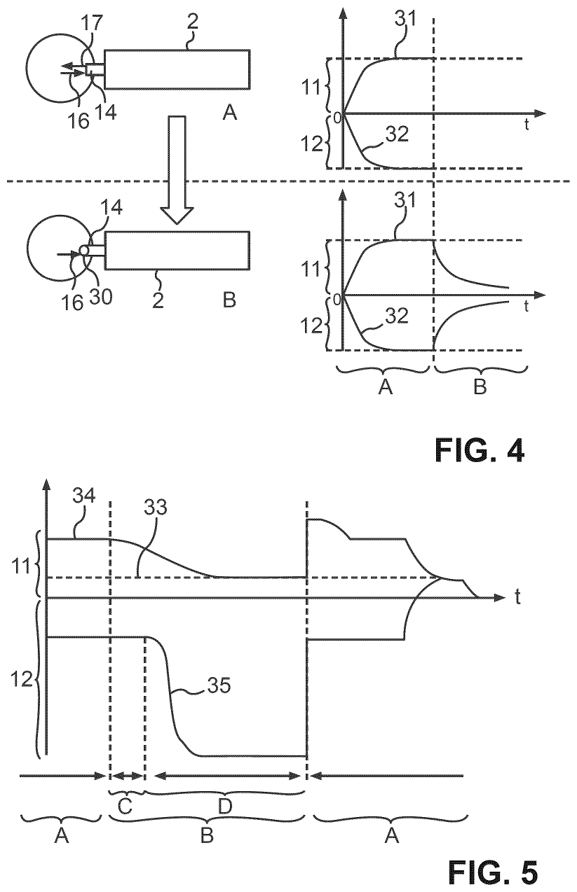

FIG. 4 describes a typical flow speed 31, 32 in relation to a time t preceding the occlusion, referred to here as situation A, and during the occlusion, referred to here as situation B. Before occlusion, the treatment fluid 16 can flow into the eye 15 and the aspiration fluid 17 can be aspirated from the eye 15. In that case, for example, contingent upon a corresponding control on the irrigation side 11 and the aspiration side 12, a predetermined target value, plotted using a dashed line, is set according to a first flow speed 31 on the irrigation side 11 and the discharge of aspiration fluid 17 up to the predetermined target value is achieved on the aspiration side 12, in accordance with a second flow speed 32. For example, no aspiration fluid 17 can be pumped out during the occlusion. Moreover, in an example, the supply of further treatment fluid 16 may be suppressed, or the supply of treatment fluid 16 may continue. Thereupon, following the detection of the occlusion, both the first flow speed 31 on the irrigation side 11 and the second flow speed 32 on the aspiration side 12 reduce and in each case approach 0.

FIG. 5 shows the profile of a set pressure p (y-axis), which may be understood to be a set irrigation-side and aspiration-side fluid pump pressure or control pressure, before the occlusion in situation A, during the occlusion in situation B and also after the end of the occlusion in a new situation A. Sketched out here are a first pressure profile 34 on the irrigation side 11, which describes the profile of a control pressure, and a second pressure profile 35 on the aspiration side 12. Further, the occlusion in situation B is divided into a partial occlusion C and a full occlusion D. It becomes clear that the pressure in the irrigation-side fluid pump 6 is started to be reduced on the irrigation side 11 during partial occlusion C so that a target pressure 33 remains set in the eye 15. This is intended to prevent an undesirably strong increase in the pressure in the eye 15. The target pressure 33 should be maintained during full occlusion D. Following the occlusion break, there is a strong, sudden increase in the pressure on the irrigation side 34 in order to compensate for a pressure drop in the eye 15 on account of a high aspiration-side pressure increase, until the target pressure 33 is reached again in the long term.

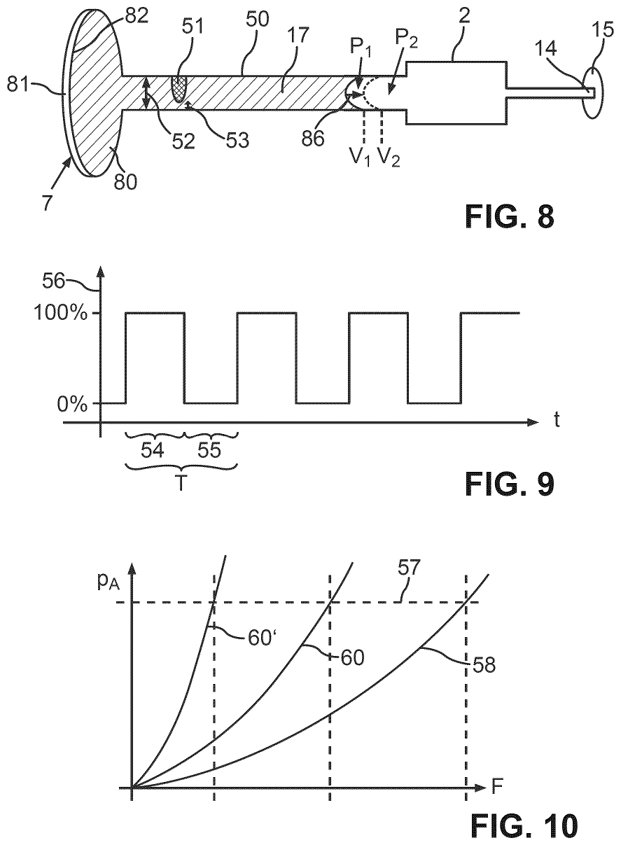

On the aspiration side 12, aspiration fluid 17 is still discharged during the partial occlusion C, and the pressure at the aspiration-side fluid pump 7 collapses in the case of the full occlusion D, when aspiration fluid 17 can no longer be pumped out owing to the closure of the needle tip 14. Following the occlusion break, the pressure p at the aspiration-side fluid pump 7 increases again until it matches the target 33. This is because the lens particle 30 no longer blocks the aspiration 50 (see reference numeral 50 in FIG. 8) and is sucked in after the occlusion break. As a consequence, there is a sudden pressure increase at the aspiration-side fluid pump 7 on account of aspiration fluid 17 flowing into the aspiration line 50.

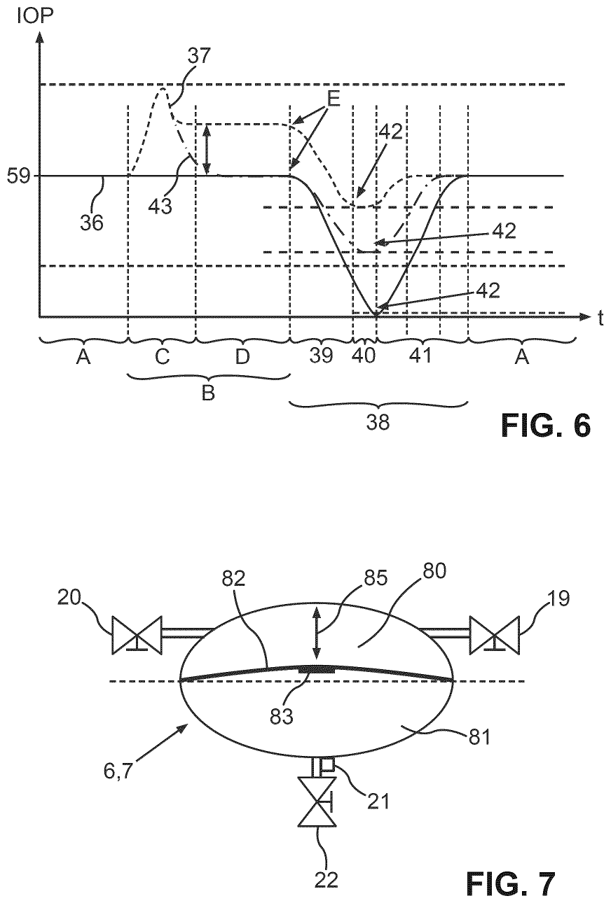

FIG. 6 shows a typical temporal profile of an intraocular pressure (IOP) in the eye 15 of a patient, referred to here as standard profile 36, and two examples of target profiles 37, 43 provided according to an exemplary embodiment of the disclosure. It becomes clear that initially before the occlusion in situation A, the standard profile 36 provides for the intraocular pressure to be set to a predetermined target intraocular pressure 59, which is patient specific, for example. Typically, attempts are made to maintain the target intraocular pressure 59, or at least to regulate back to said target intraocular pressure, during the occlusion and hence in situation B. The increase in the intraocular pressure at the start of the occlusion in situation B, shown for the target profile 37, is thus typically reduced in such a way that the predetermined target intraocular pressure 59 is reached again. This is shown here by way of example for the target profile 43.

Starting from the occlusion break, plotted here as a situation E, a particularly low intraocular pressure is achieved in the standard profile 36 since a large amount of aspiration fluid 17 is suddenly aspirated quickly from the eye 15 on the aspiration side 12. In this case, the target intraocular pressure 59 is again reached once a time period 38, composed of a sensory detection time 39 for detecting the occlusion break, a delay time 40 of an occlusion break detection algorithm and a response time 41 of the irrigation side 11 to the occlusion break for example, has elapsed.

The disclosure described below is intended to ensure that one of the target profiles 37, 43 is set. In this case, a minimum value 42 of the standard profile 36 is increased to the minimum value 42 of the respective target profile 37, 43. Overall, a lower pressure drop of the intraocular pressure following the occlusion break is observed as result, when compared with the standard profile 36. In the target profile 37, a target intraocular pressure increase is determined and taken into account during the occlusion, i.e., the predetermined target intraocular pressure 59 is not set during the occlusion; instead, an intraocular pressure corresponding to the target intraocular pressure 59 plus the determined target intraocular pressure increase is set. The difference between the target intraocular pressure 59, and hence the target profile 43 on the one hand, and the intraocular pressure set according to the target profile 37 on the other hand is accentuated here with a double-headed arrow.

FIG. 7 shows a detailed illustration of a fluid pump 6, 7, for example the aspiration-side fluid pump 7. It becomes clear that the fluid pump 6, 7 includes a pump chamber 80 and a drive chamber 81. The drive chamber 81 is coupled to the compressed-air valve 22. The position sensor 21 can be used to detect a movement of the separation element 82 between the pump chamber 80 and the drive chamber 81, for which purpose for example a metal plate 83 is positioned in the separation element 82 and its movement is detected with the position sensor 21. For example, the change in movement is accentuated here as deflection 85.

According to an exemplary embodiment of the disclosure, provision is made for an occlusion of the needle tip 14 to be detected by applying an occlusion detection algorithm. In order to detect the occlusion, the occlusion detection algorithm can be applied to movement information, for example, which describes a current movement of the separation element 82 between the pump chamber 80 and the drive chamber 81 of the at least one aspiration-side fluid pump 7. The occlusion is detected at least on the basis of the movement information being smaller than a predetermined movement limit value. After the occlusion is detected, at least one measure is taken in order to reduce a volume flow of aspiration fluid 17 from the eye 15 to the at least one aspiration-side fluid pump 7 that occurs after the detected occlusion breaks, in comparison with the volume flow of aspiration fluid 17 without a measure being taken. Three different measures, which are distinguished as measure a, measure b and measure c, are described below. A plurality of measures a, b, c may be taken simultaneously or successively. When the respective measure a, b, c is taken, the respective measure is, e.g., activated and/or performed.

FIG. 8 shows an example of measure a. The handpiece 2 and the at least one aspiration-side fluid pump 7 are shown. The aspiration line 50, along which the aspiration fluid 17 is aspirated from the eye 15 via the handpiece 2 to the at least one aspiration-side fluid pump 7 if there is no occlusion present, is shown between the needle tip 14 of the handpiece 2 and the at least one aspiration-side fluid pump 7. According to measure a, at least one volume flow limiting element 51, which is encompassed by the ophthalmic surgical system 1, is provided. The at least one volume flow limiting element 51 is configured to be moved from a normal position into a measure position. The measure position is shown in FIG. 8. Measure a provides for the volume flow limiting element 51 to be moved into the measure position and kept in the measure position.

In the normal position, a portion of the ophthalmic surgical system 1 through which the aspiration fluid 17 can flow between the handpiece and the at least one aspiration-side fluid pump, with the portion through which fluid can flow for example being the aspiration line 50, has a first cross section 52, which may be referred to as cross section A1. In the normal position, no portion of the volume flow limiting element 51 for example protrudes into the portion through which fluid can flow, for example into the aspiration line 50. In the measure position, the portion though which fluid can flow, for example the aspiration line 50, has at least locally a second cross section 53 that is reduced in comparison with the first cross section 52. In an exemplary embodiment, the second cross section 53 is predetermined in such a way that the portion through which fluid can flow, in particular the aspiration line 50, is reduced at least locally by 20 percent to 100 percent, in particular by 70 percent to 90 percent, in the measure position, when compared with the normal position, and hence closed. An example is shown here, in which the second cross 53 is chosen in such a way that the aspiration line 50 is closed between 70 percent and 90 percent, i.e., a degree of closure between 70 percent and 90 percent is realized. The at least one volume flow limiting element 51 may be embodied as a valve, for example, which can be moved into the aspiration line 50. The second cross section 53 may be predetermined on the basis of the handpiece 2, in particular a diameter of the needle tip 14, and/or a pressure in the at least one aspiration-side fluid pump 7.

Labelled with a volume V1 and a pressure p1, FIG. 8 illustrates a situation in which the occlusion has occurred. After the occlusion break, the volume of aspiration fluid 17 for example increases to the volume V2, plotted with dashed lines, at a pressure p2. This increase in the volume of aspiration fluid 17 in the aspiration line 50 is plotted here with a directional arrow 86.

Measure b provides for at least one volume flow limiting element 51, for example the volume flow limiting element 51 from FIG. 8, to be moved repeatedly back and forth between the normal position and the measure position. In the measure position, the second cross 53 is 0, i.e., the volume flow of aspiration fluid 17 through the aspiration line 50 is completely prevented.

FIG. 9 elucidates measure b on the basis of a temporal profile of the switch between the normal position and the measure position. Here, time t is plotted on an x-axis and a degree of closure 56 of the at least one volume flow limiting element 51 is plotted on a y-axis. The at least one volume flow limiting element 51 is in the normal position in the case of a degree of closure 56 of 0 percent, whereas it is in the measure position in the case of the degree of closure 56 of 100 percent, under the assumption that the second cross section 53 is 0 and thus the aspiration line 50 is completely blocked locally.

Provision may be made for the at least one volume flow limiting element 51 to be repeatedly moved between the normal position and the measure position in such a way that it is alternately arranged in the measure position for a predetermined first time period 54 and, thereafter, in the normal position for a predetermined second time period 55. This back-and-forth movement is repeated again and again. The first time period 54 and the second time period 55 together result in a period duration T. The first time period 54 and the second time period 55 may have the same length or different lengths. In order to reduce the volume flow of aspiration fluid 17 particularly strongly, first time periods 54 which are as long as possible and, in comparison therewith, short second time periods 55 may for example be chosen, i.e., the volume flow limiting element 51 may for example be in the measure position for as long as possible and as frequently as possible, with for example all volume flow through the aspiration line 50 being suppressed in said measure position. Other configurations of the first time period 54 and the second time period 55 are possible.

The first time period 54 and/or the second time period 55 may be predetermined on the basis of the handpiece 2 and/or the pressure in the at least one aspiration-side fluid pump 7, i.e., they may have analogous dependencies like the second cross section 53.

FIG. 10 elucidates a relationship between the volume flow, which is plotted here, abbreviated to the letter F, on the x-axis, and an aspiration pressure p_{A}, i.e., a pressure present in the aspiration-side fluid pump 7, which is plotted on the y-axis. An exemplary aspiration pressure 57 of the utilized aspiration-side fluid pump 7, which could prevail in the event of an occlusion break, is plotted. It becomes clear from the plotted profile 58 that relatively high volume flows are expected after the occlusion break, but the modified profiles 60, 60′, for which significantly smaller volume flows are obtained, can be obtained should measure a and/or measure b be taken. The measure a and/or the measure b thus makes it possible to successfully reduce the aspiration-side volume flow. Thus, the profile 58 is modified to one of the profiles 60, 60′ or to a profile between the profile 58 and the profile 60′ by virtue of taking, e.g., measure a and/or measure b.

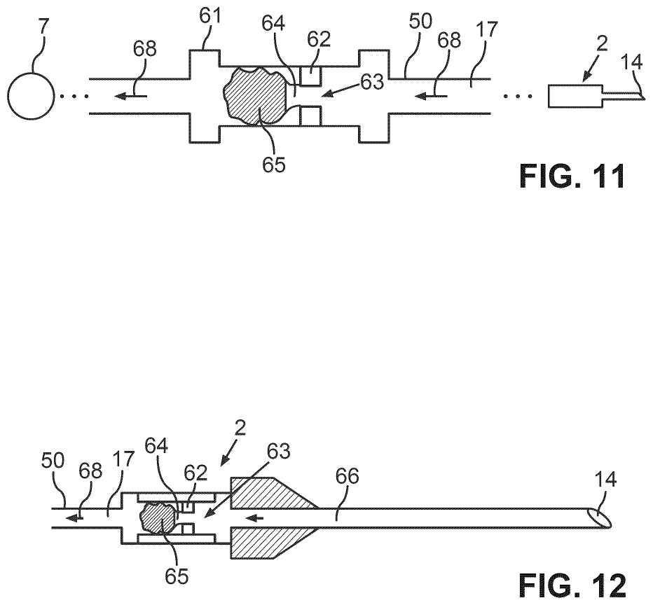

FIG. 11 shows an example of measure c. In measure c, the aspiration fluid 17 is guided through at least one flow restriction means 62, which is arranged between the needle tip 14 of the handpiece 2 and the at least one aspiration-side fluid pump 7. The flow restriction means 62 is configured to bring about a specific pressure drop in the volume flow of the aspiration fluid 17 downstream of the at least one flow restriction means 62, as seen in the flow direction 68 of the aspiration fluid 17, in the case of the occlusion break. Cavitation occurs when the specific pressure drop is brought about, i.e., cavitation 65 can be observed. Here, this occurs in the plotted pressure drop region 64. A region of constriction 63, through which the aspiration fluid 17 flows or passes, is achieved by way of the flow restriction means 62.

In the shown example, the flow restriction means 62 is arranged in the aspiration line 50. In this case, the flow restriction means 62 is integrated into an add-on component 61 which for example is installed in a tube between the handpiece 2 and the aspiration-side fluid pump 7.

FIG. 12 shows an example of measure c in which the flow restriction means 62 is integrated into the handpiece 2. In this case, the flow restriction means 62 is thus situated in the vicinity of the needle tip 14 and far from the aspiration-side fluid pump 7 by comparison.

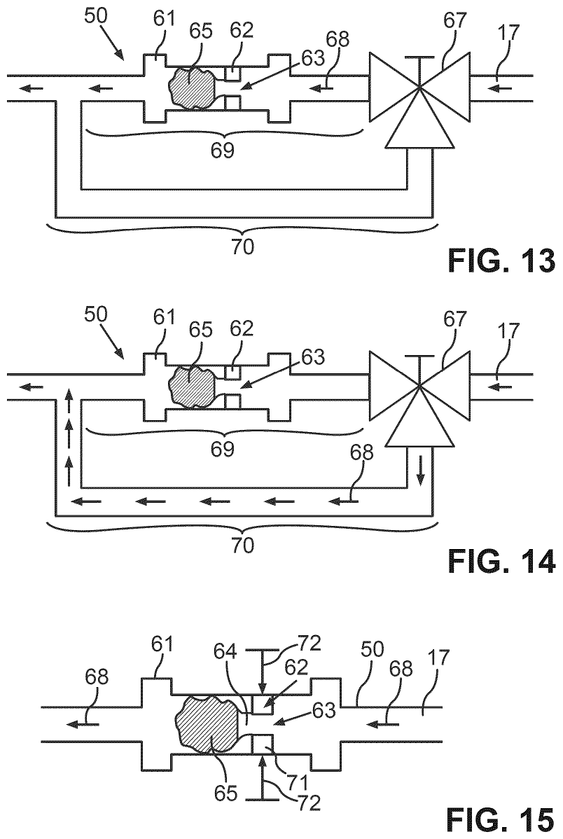

FIG. 13 and FIG. 14 show a further example of the configuration of the flow restriction means 62. The ophthalmic surgical system 1 may include at least one diverting element 67, which for example is embodied as a directional control valve. When measure c is taken, controlling the at least one diverting element 67 ensures that the aspiration fluid 17 is guided through a first partial flow path 69 of the ophthalmic surgical system 1 in which the flow restriction means 62 is arranged. This is accentuated here by the flow direction 68 of the aspiration fluid 17. When measure c is not taken, the diverting element 67 is controlled such that the aspiration fluid 17 is guided through a second partial flow path 70 of the ophthalmic surgical system 1 that differs from the first partial flow path 69, without a flow restriction means 62 being arranged in the second partial flow path 70. This is shown in FIG. 14, in which corresponding flow directions 68 of the aspiration fluid 17 are plotted.

FIG. 15 shows a further exemplary embodiment in which the flow restriction means 62 includes a movable stop 71. The latter may be moved into a predetermined position or may be located in the predetermined position when measure c is taken. In the predetermined position, the pressure drop at which cavitation happens occurs downstream of the flow restriction means 62, as viewed in the flow direction 68, after the occlusion break. Stop movement directions 72 of the movable stop 71 during the movement into the predetermined position are plotted here with the aid of arrows. The movable stop 71 allows the region of constriction 63 to be configured dynamically such that the movable stop 71 may for example be completely retracted or pulled back during a surgical operation without an occlusion event. A cross section in the area of the region of constriction 63 may then correspond to the first cross section 52 in FIG. 8, for example.

In general, the flow restriction means 62 may be arranged in the handpiece 2, in the aspiration line 50, in the console 3 upstream of the at least a portion of the at least one aspiration-side fluid pump 7, as seen in the flow direction 68 of the aspiration fluid 17, and/or in the cassette 5 upstream of the at least a portion of the at least one aspiration-side fluid pump 7, as seen in the flow direction 68 of the aspiration fluid 17.

After the detection of the occlusion, provision may be made for the at least one aspiration-side fluid pump 7 to be controlled in such a way that a negative pressure, for example a maximum possible negative pressure, sets in in the at least one aspiration-side fluid pump 7. After the detection of the occlusion break, the at least one aspiration-side fluid pump 7 is controlled in such a way that a target intraocular pressure 59 in accordance with the surgical operation without an occlusion event sets in. The respective measure taken, at least measure a and/or measure b, may be terminated no later than when the target intraocular pressure is reached, for example already immediately after the detection of the occlusion break. In the context of terminating measure c, the movable stop 71 may for example be moved back again from the predetermined position into an initial position and/or the diverting element 67 may for example be controlled in such a way that the aspiration fluid 17 flows along the second partial flow path 70.

The occlusion break may be applied, for example, by applying an occlusion break detection algorithm to the aforementioned movement information. For example, the occlusion break may be detected by virtue of the movement information being larger than the predetermined movement limit value. For example, a detection time for detecting the occlusion break may be between 50 and 100 milliseconds.

Overall, the examples show complete avoidance of eye collapse when using a handpiece 2 with any low pressure loss as a result of dynamically increasing the pressure loss in the aspiration-side fluid path in the case of occlusion by reducing the aspiration-side valve degree of opening of the valves upstream of the inlet of the aspiration-side pump diaphragm while simultaneously improving the fluid performance in the non-occluded surgical operation.

Measure a may include that pressure losses in the aspiration-side fluid path, i.e., on the aspiration side 12, are increased significantly by virtue of the valve, i.e., the volume flow limiting element 51, being closed, for example to an extent of 90 percent, upstream of the active pump diaphragm, i.e., between eye 15 and aspiration-side fluid pump 7. Increasing the pressure losses of the valves by increasing the degree of closure is utilized, for example, in order to reduce the volume flow from the eye 15 in the direction of the aspiration diaphragm to a minimum in the event of an occlusion break and simultaneously continue to enable occlusion break detection by way of a recommencing diaphragm movement.

The movement of the diaphragm, i.e., the separation element 82, can be used for the detection of the occlusion break. As soon as the occlusion breaks, the aspiration fluid 17 flows from the eye 15 into the tube on the aspiration side 12, i.e., the aspiration line 50, and, at a given air pressure in the drive chamber 81, leads to a movement of the diaphragm, i.e., of the separation element 82. Since all that is decisive for the occlusion break detection is a fluidic pressure change, i.e., the propagation of a pressure change in the tube, the detection in the case of a still closed valve, i.e., in the case of the closed volume flow limiting element 51, is just as fast as in the case of a fully open valve or volume flow limiting element 51. The procedure for minimizing the intraocular pressure drop after an occlusion break is described below:
1. Detecting the active pump diaphragm that applies the vacuum, i.e., generates the negative pressure. The valve upstream of the non-active pump diaphragm, i.e., the inlet valve 19, is closed completely, i.e., a non-utilized aspiration-side fluid pump 7 is temporarily separated from the handpiece 2.
2. Waiting until an occlusion is detected.
3. After the occlusion is detected, the maximum vacuum possible by regulation, i.e., a maximum negative pressure, is applied to the aspiration-side pump diaphragm, i.e., to the fluid pump 7. The maximum suction force acts on the occluding lens particle 30 and hence assists the occlusion break.
4. The opening of the valve, i.e., of the volume flow limiting element 51, between the eye 15 and the active pump diaphragm, i.e., the aspiration-side fluid pump 7, is reduced to a degree of closure of for example z=90 percent by virtue of being moved into a corresponding measure position. As a result of the closure, the pressure losses in the aspiration-side fluid path are greatly increased and, as a result, the intraocular pressure drop after the occlusion break is reduced.
5. As a result of the applied vacuum, i.e., the negative pressure set, a vacuum, i.e., a negative pressure, in the aspiration tube, i.e., in the aspiration line 50, is nearly just as strong as at the pump diaphragm, i.e., the aspiration-side fluid pump 7. If an occlusion break occurs, a certain amount of liquid, i.e., aspiration fluid 17, flows from the eye 15 into the tube, i.e., into the aspiration line 50, as a result of the vacuum, i.e., negative pressure, in the aspiration tube, i.e., in the aspiration line 50.
While the pressure wave is still propagating and before the fluidic system can react to the detected occlusion break, the volume flow of aspiration fluid 17 flowing out of the eye 15 is reduced since, firstly, the aspiration-side pressure loss is high as a result of the closed valve, i.e., the closed volume flow limiting element 51, and a high pressure loss opposes the flowing-in aspiration fluid 17 and since, secondly, the vacuum, i.e., the negative pressure, in the tube, i.e. in the aspiration line 50, is greatly reduced due to the aspiration fluid 17 flowing in. The pressure drop (surge), i.e., the significant intraocular pressure drop, is attenuated. As a result, the volume flow out of the eye 15 is additionally reduced and the extent of the pressure drop is reduced.
6. After the occlusion break has been detected, a reflux movement of the active aspiration pump diaphragm can set the vacuum, i.e., the negative pressure, in accordance with the regular surgical operation, i.e., the pump pressure before the occlusion.
7. Subsequently, the valves, i.e., the volume flow limiting element 51 and the inlet valve 19, are fully opened again. The application of a vacuum, i.e., negative pressure, is continued according to the surgical operation without an occlusion event.

Measure b provides a technical method of how the response of the fluidic system can be controlled or regulated algorithmically in order to minimize the extent of pressure drop and enable a health-preserving operation for the patient. As a result of better maintenance of the target intraocular pressure 59, the anterior chamber volume in the eye can be kept stable, and the stress for the optic nerve and endothelial cells and the risk of an anterior chamber collapse in the eye can be prevented. To this end, a dynamic increase in the aspiration-side pressure losses in the case of occlusion is performed by quickly and cyclically opening and closing the respective valve, i.e., the volume flow limiting element 51, upstream of the active pump diaphragm, i.e., of the aspiration-side fluid pump 7, i.e., the movement between the normal position and the measure position.

As a result of the inertia of the liquid flowing in, i.e., the aspiration fluid 17, the pressure loss increases relatively uniformly despite the cyclic opening and closing. The pressure loss depends on the periodically repeating closing and opening duration of the valve, i.e., of the volume flow limiting element 51, i.e., on the first time period 54 and the second time period 55. Compared with the reduction in the valve degree of opening provided in measure a for increasing the aspiration-side pressure losses, the rapid opening and closing of the valves, i.e., of the volume flow limiting element 51, however offers the advantage that the residual open area of the valve and hence of the volume flow limiting element 51 cannot be blocked by removed lens particles 30. Cyclic opening and closing begins after an occlusion has been detected.

The period duration T describes the duration of a repeating opening and closing cycle. For example, T_{w} or Tₒₚₑₙ or Tₒₙ describes the time period in which the volume flow limiting element 51 is open, i.e., the first time period 54. For example, T_{closed} or T_{off} describes the closing time period, i.e., the second time period 55. The ratio of T_{w} to T corresponds to a duty cycle (DC) in a manner analogous to pulse width modulation. In this case, the frequency f of a periodic cycle is defined as 1/T.

In this case, the pressure loss can be increased both by a reduction in the duty cycle and an increase in the frequency f. In general, attempts should be made to reduce the duty cycle since the valves, i.e., the volume flow limiting element 51, need to be actuated less often as a result.

For example, the volume flow limiting element 51 can be actuated with compressed air. Every movement of the volume flow limiting element 51 requires a predetermined amount of compressed air, which in turn is provided by a compressor; the latter requires the use of electrical energy and may adversely affect the system efficiency of the ophthalmic surgical system 1.

This possible solution in the case of an occlusion break is described below such that the pressure drop can be channeled by preventive measures even before the actual detection time 39 has elapsed, and hence the extent of the pressure drop, i.e., of the intraocular pressure drop, can be damped and critical intraocular pressure ranges can be prevented:
1. Detecting the occlusion on the basis of the membrane movement. After occlusion is detected, the maximum vacuum possible by regulation, i.e., the negative pressure, is applied to the aspiration-side pump diaphragm, i.e., to the fluid pump 7. The maximum suction force acts on the occluding lens particle 30 and hence assists an occlusion break.
2. Monitoring the active pump diaphragm which applies the vacuum. The inlet valve 19 upstream of the inactive pump diaphragm is closed completely. The pressure loss is increased by cyclically closing and opening the valve, i.e., the volume flow limiting element 51, upstream of the active pump diaphragm, i.e., the aspiration-side fluid pump 7. The required increase in the pressure loss as a result of the closing and opening cycle (characterized duty cycle and frequency) is selected from a characteristic map by virtue of the pressure loss curve of the fluid path initially being subtracted from the required pressure loss curve for a meaningful pressure drop behavior. The difference then corresponds to the required pressure loss increase. The two associated parameters DC and f are taken from a characteristic map. The pressure drop behavior is improved significantly by increasing the pressure losses of the aspiration-side fluid path.
3. As a result of the applied vacuum, i.e., the negative pressure, a vacuum in the aspiration tube, i.e., in the aspiration line 50, is nearly just as strong, or a negative pressure therein is almost the same, as at the pump diaphragm, i.e., the aspiration-side fluid pump 7. If an occlusion break occurs, a certain amount of aspiration fluid 17 flows from the eye 15 into the tube, i.e., into the aspiration line 50, as a result of the vacuum in the aspiration tube, i.e., in the aspiration line 50, i.e., as a result of the negative pressure there. While the pressure wave is still propagating and before the fluidic system can react to the detected occlusion break, the volume flow flowing out of the eye 15 is reduced since, firstly, the aspiration-side pressure loss is high as a result of the fast, cyclic closing and opening of the valve, i.e., of the volume flow limiting element 51, and a high pressure loss opposes the flowing-in aspiration fluid 17 and, secondly, the vacuum, i.e., the negative pressure, in the tube, i.e., in the aspiration line 50, is greatly reduced due to the aspiration fluid 17 flowing in. The pressure drop is attenuated. As a result, the volume flow out of the eye 15 is additionally reduced and the extent of the pressure drop is reduced significantly.
4. After the occlusion break has been detected, a reflux movement of the active aspiration pump diaphragm, i.e., the aspiration-side fluid pump 7, can set the vacuum, i.e., the negative pressure, in accordance with the regular surgical operation, i.e., the pump pressure before the occlusion. The movement of the diaphragm, i.e., the separation element 82, is used for the detection of the occlusion break. As soon as the occlusion breaks, the aspiration fluid 17 flows from the eye 15 into the tube, i.e., the aspiration line 50, and, at a given air pressure, leads to a movement of the diaphragm.
5. Subsequently, for example after the target intraocular pressure 59 has been set again, the valve, i.e., the volume flow limiting element 51, of the active pump diaphragm, i.e., of the aspiration-side fluid pump 7, is opened completely again. The application of a vacuum, i.e., negative pressure, is continued corresponding to the surgical operation without an occlusion event.

Measure c provides for the use of the flow restriction means 62 in the fluid path between the eye 15 and aspiration-side pump apparatus, i.e., the fluid pump 7, for the targeted volume flow limitation by cavitation 65 in the case of a pressure drop. In this case, the flow restriction means 62 does not serve in the typical sense as a flow restriction apparatus for locally increasing the pressure but as a volume flow limiting apparatus as a result of the defined and targeted formation of vapor with cavitation 65 as a result of the volume-flow-dependent pressure drop on the outflow side. A flow restriction means 62 typically describes a cross-sectional constriction in a fluid-guiding line. There is a pressure increase on the inflow side as a result of the constriction, and there is a pressure drop of equal absolute value on the outflow side. If the pressure drop on the outflow side leads to an undershooting of the vapor pressure, a vapor pressure region arises downstream of the flow restriction means 62, i.e., the cavitation 65. This process is referred to as fluid column separation. Additionally, the pressure difference across the flow restriction means 62 also leads to the flowing aspiration fluid 17 being delayed continuously.

The physical phenomenon of cavitation 65 in this case describes the formation of vapor-filled cavities in the aspiration fluid 17 if the vapor pressure is undershot. In the case of a flow restriction means 62, a fixed upstream pressure initially leads to an increasing mass flow with a decreasing backpressure. If cavitation 65 sets in, it remains constant. The effect can advantageously be used in the case of an occlusion break by virtue of a skillful choice for the cross section of the flow restriction means allowing the pressure on the outflow side to drop in such a way that there is a targeted onset of cavitation 65 and thus a volume flow limitation for a defined volume flow. The choice of volume flow limitation should be taken in such a way here that the surge depth or pressure drop depth can still be reliably compensated. What is advantageous here is that the regions upstream and downstream of the flow restriction means 62 are hydraulically decoupled from each other as a result of the cavitation 65. That is to say, pressure fluctuations upstream and downstream of the flow restriction means 62 are not noticeable (over time) in the absolute value of the integral mass flow for as long as the aspiration fluid 17 cavitates in the flow restriction means 62 or directly downstream of the flow restriction means 62. Applied to the application of cataract surgery and fluidics of the eye 15, the intraocular pressure can thus be kept virtually constant in the case of a pressure drop by virtue of said intraocular pressure still being maintained by the irrigation-side pump apparatus, i.e., the fluid pump 7, in the case of a constant volume flow. In parallel, an occlusion break can continue to be detected without being influenced by using the mass flow. The flow restriction means 62 thus serves as a passive safety element which prevents a collapse of the eye 15 as a result of a significant pressure drop, for example independently of a chosen diameter of the hollow needle 88 of the handpiece 2. The flow restriction means 62 may be used independently of the system.

The following technical implementation variants of the flow restriction means 62 are possible:
1. The flow restriction means 62 is introduced between the handpiece 2 and the aspiration-side fluid pump 7. In this case, the flow restriction means 62 may be integrated into the tube, i.e., into the aspiration line 50, downstream of the handpiece 2 or may be plugged on as a disposable component.
2. Integration of the flow restriction means 62 into the handpiece 2.
3. Switchable diversion, i.e., the diverting element 67, which is switched in the case of occlusion. In this case, integration is into the handpiece, the aspiration tube or into the cassette 5 upstream of the pump diaphragm, i.e., of the aspiration-side fluid pump 7.

4. A flow restriction means 62 with adjustable cross section. The flow restriction means 62 may be configured with an adjustable stop 71. The desired volume flow limitation may be defined by adjusting the stop 71.

A combination of the four variants mentioned may be provided.

In other words, the disclosure relates to an ophthalmic surgical system 1 for treating an eye 15, having an aspiration-side fluid pump 7 for conveying an aspiration fluid 17 and a handpiece 2, at least a portion of said handpiece being introducible into the eye 15 with a needle tip 14 and said handpiece being coupled to the aspiration-side fluid pump 7. The ophthalmic surgical system 1 is capable of detecting an occlusion at the needle tip 14; and then taking at least one measure in order to reduce the volume flow of aspiration fluid 17 that occurs after an occlusion break. At least one of following measures a, b, and/or c can be taken:
a) a volume flow limiting element 51 is moved from a normal position into a measure position and kept there, and/or
b) a volume flow limiting element 51 is moved repeatedly back and forth between the normal position and the measure position, and/or
c) the aspiration fluid 17 is guided through at least one flow restriction means 62 which brings about a pressure drop in the volume flow downstream of the at least one flow restriction means 62 in the case of an occlusion break, and so cavitation occurs.

### LIST OF REFERENCE NUMERALS

1 Ophthalmic surgical system
2 Handpiece
3 Console
4 Cassette accommodation region
5 Cassette
6 Irrigation-side fluid pump
7 Aspiration-side fluid pump
8 Treatment fluid storage vessel
9 Treatment fluid line
10 Supply line
11 Irrigation side
12 Aspiration side
13 User interface
14 Needle tip
15 Eye
16 Treatment fluid
17 Aspiration fluid
18 Port
19 Inlet valve
20 Outlet valve
21 Position sensor
22 Compressed-air valve
23 Aspiration fluid collection vessel
24 Clamp
25 Valve
26 Pressure sensor
30 Lens particle
31 First flow speed
32 Second flow speed
33 Target pressure
34 First pressure profile
35 Second pressure profile
36 Standard profile
37 Target profile
38 Time period
39 Detection time
40 Delay time
41 Response time
42 Minimum value
43 Target profile
50 Aspiration line
51 Volume flow limiting element
52 First cross section
53 Second cross section
54 First time period
55 Second time period
56 Degree of closure
57 Pump characteristic curve
58 Profile
59 Target intraocular pressure
60 Profile
61 Add-on component
62 Flow restriction means
63 Region of constriction
64 Pressure drop region
65 Cavitation
67 Diverting element
68 Flow direction
69 First partial flow path
70 Second partial flow path
71 Stop
72 Stop movement directions
80 Pump chamber
81 Drive chamber
82 Separation element
83 Metal plate
85 Deflection
86 Directional arrow
87 Sleeve
88 Hollow needle

What is claimed is:

1. An ophthalmic surgical system for treating an eye, the ophthalmic surgical system comprising:
a console including at least one portion of at least one aspiration-side fluid pump configured to convey an aspiration fluid; and
an ophthalmic surgical handpiece configured to treat the eye, having a hollow needle with a needle tip, at least a portion of the ophthalmic surgical handpiece being introducible into the eye with the needle tip of the hollow needle of the handpiece, and being coupled at least indirectly to the aspiration-side fluid pump;
wherein the ophthalmic surgical system is configured to:
detect an occlusion at the needle tip by applying an occlusion detection algorithm,
take at least one measure after a detection of the occlusion to reduce a volume flow of aspiration fluid from the eye to the at least one aspiration-side fluid pump that occurs after the detected occlusion breaks, in comparison with the volume flow of aspiration fluid without a measure being taken,
take at least one of following measures (a), (b), and/or (c) as the at least one measure:
(a) move at least one volume flow limiting element from a normal position into a measure position and keep the at least one volume flow limiting element in the measure position, wherein a portion of the ophthalmic surgical system, through which the aspiration fluid can flow, has, in a normal position, a first cross section between the ophthalmic surgical handpiece and the at least one aspiration-side fluid pump and, in the measure position, a second cross section which is at least locally reduced in comparison with the first cross section, the second cross section being predetermined based on the ophthalmic surgical handpiece and/or a pressure in the at least one aspiration-side fluid pump, and/or
(b) move at least one volume flow limiting element repeatedly back and forth between the normal position and the measure position, and/or
(c) guide the aspiration fluid through at least one flow restriction means arranged between the needle tip of the ophthalmic surgical handpiece and the at least one aspiration-side fluid pump, the flow restriction means being configured to bring about, in case of the occlusion break, a pressure drop in the volume flow of aspiration fluid downstream of the at least one flow restriction means, as seen in the flow direction of the aspiration fluid, and a cavitation occurring in the case of the brought-about pressure drop.

2. The ophthalmic surgical system according to claim 1, wherein when measure (a) is taken, the second cross section is predetermined such that, in the measure position, the portion of the ophthalmic surgical system through which the aspiration fluid can flow is closed at least locally by 20 percent to 100 percent, or by 70 percent to 90 percent, in comparison with the normal position.

3. The ophthalmic surgical system according to claim 1, wherein when measure (b) is taken, the ophthalmic surgical system is configured to repeatedly move the at least one volume flow limiting element between the normal position and the measure position such that the at least one volume flow limiting element is alternately arranged in the normal position for a predetermined first time period and, thereafter, in the measure position for a predetermined second time period.

4. The ophthalmic surgical system according to claim 3, wherein the predetermined first time period and the predetermined second time period are predetermined based on the ophthalmic surgical handpiece and/or a pressure in the at least one aspiration-side fluid pump.

5. The ophthalmic surgical system according to claim 1, wherein when measure (c) is taken, the flow restriction means is arranged in the ophthalmic surgical handpiece and/or in an aspiration line and/or in the console upstream of the at least a portion of the at least one aspiration-side fluid pump, as seen in a flow direction of the aspiration fluid, and/or in a cassette of the ophthalmic surgical system, the cassette including at least a portion of the at least one aspiration-side fluid pump, upstream of the at least a portion of the at least one aspiration-side fluid pump, as seen in the flow direction of the aspiration fluid.

6. The ophthalmic surgical system according to claim 1, wherein for measure (c), the ophthalmic surgical system further comprises at least one diverting element,
wherein the ophthalmic surgical system is configured, when measure (c) is taken, to guide the aspiration fluid through a first partial flow path of the ophthalmic surgical handpiece, in which the flow restriction means is arranged, by controlling the at least one diverting element, and
wherein the ophthalmic surgical system is configured, when measure (c) is not taken, to guide the aspiration fluid through a second partial flow path of the ophthalmic surgical handpiece which differs from the first partial flow path and in which no flow restriction means is arranged.

7. The ophthalmic surgical system according to claim 1, wherein for measure (c), the flow restriction means comprises a movable stop and is configured, when taking measure (c), to move the movable stop into a predetermined position in which, in case of the occlusion break, the cavitation occurs during the brought-about pressure drop.

8. The ophthalmic surgical system according to claim 1, further configured to:
after the detection of the occlusion, control the at least one aspiration-side fluid pump such that a negative pressure, or a maximum possible negative pressure, sets in in the at least one aspiration-side fluid pump; and
after the detection of the occlusion break, control the at least one aspiration-side fluid pump such that a target intraocular pressure in the eye, which is predetermined for a surgical operation without an occlusion event, sets in and, no later than when the target intraocular pressure is reached, terminate at least taken measure (a) and/or taken measure (b).

9. The ophthalmic surgical system according to claim 1, further configured to:
apply the occlusion detection algorithm to movement information describing a current movement of a separation element between a pump chamber and a drive chamber of the at least one aspiration-side fluid pump to detect the occlusion at least based on the movement information being smaller than a predetermined movement limit value, and/or
apply an occlusion break detection algorithm to movement information describing a current movement of a separation element between a pump chamber and a drive chamber of the at least one aspiration-side fluid pump to detect the occlusion break at least based on the movement information being larger than a predetermined movement limit value.

10. A method for operating an ophthalmic surgical system having at least a console which includes at least one portion of at least one aspiration-side fluid pump configured to convey an aspiration fluid, and an ophthalmic surgical handpiece having a hollow needle with a needle tip, at least a portion of the ophthalmic surgical handpiece being introducible into an eye with the needle tip of the hollow needle of the ophthalmic surgical handpiece, and the ophthalmic surgical handpiece being coupled at least indirectly to the aspiration-side fluid pump, the method comprising:
taking at least one measure after a detection of an occlusion at the needle tip to reduce a volume flow of aspiration fluid from the eye to the at least one aspiration-side fluid pump that occurs after the occlusion breaks, in comparison with the volume flow of aspiration fluid without a measure being taken,
wherein at least one of following measures (a), (b), and/or (c) is taken as the at least one measure:
(a) moving at least one volume flow limiting element from a normal position into a measure position and keeping the at least one volume flow limiting element in the measure position, wherein a portion of the ophthalmic surgical system through which the aspiration fluid can flow has, in the normal position, a first cross section between the ophthalmic surgical handpiece and the at least one aspiration-side fluid pump and, in the measure position, a second cross section that is at least locally reduced in comparison with the first cross section, wherein the second cross section is predetermined based on the ophthalmic surgical handpiece and/or a pressure in the at least one aspiration-side fluid pump; and/or
(b) repeatedly moving at least one volume flow limiting element back and forth between the normal position and the measure position; and/or
(c) guiding the aspiration fluid through at least one flow restriction means arranged between the needle tip of the ophthalmic surgical handpiece and the at least one aspiration-side fluid pump, wherein the flow restriction means brings about, in the case of the occlusion break, a pressure drop in the volume flow of aspiration fluid downstream of the at least one flow restriction means, as seen in the flow direction of the aspiration fluid, with a cavitation occurring in the case of the brought-about pressure drop.
